(19) United States
(12) Patent Application Publication
(10) Pub. No.: US 2007/0254968 A1
(43) Pub. Date: Nov. 1, 2007

(54) METHOD OF DELIVERY, REPLACEMENT, AND REMOVAL OF FISCHER-TROPSCH CATALYST

(75) Inventors: Kenneth Agee, Bixby, OK (US); Kym B. Arcuri, Tulsa, OK (US)

Correspondence Address: BAKER & MCKENZIE LLP, Pennzoil Place, South Tower, 711 Louisiana, Suite 3400, HOUSTON, TX, 77002-2716, US

(73) Assignee: SYNTROLEUM CORPORATION, Tulsa, OK (US)

(21) Appl. No.: 11/741,118

(22) Filed: Apr. 27, 2007

Related U.S. Application Data
(60) Provisional application No. 60/795,448, filed on Apr. 27, 2006.

(51) Int. Cl. C07C 27/26

(52) U.S. Cl. 518/726

(57) ABSTRACT

A method for delivery, replacement and removal of Fischer-Tropsch catalyst from a gas-to-liquid (GTL) facility utilizing a Fischer-Trosch reaction. Embodiments also relate to a method for delivery, replacement and removal of Fischer-Tropsch catalyst from a transportable GTL facility.

### CROSS REFERENCE TO RELATED APPLICATIONS

This application claims priority to U.S. Provisional Application No. 60/795,448, filed Apr. 27, 2006, which is incorporated herein in its entirety.

### FEDERALLY SPONSORED RESEARCH

Not applicable.

### REFERENCE TO MICROFICHE APPENDIX

Not applicable.

### FIELD OF THE INVENTION

This invention relates to a method for delivery, replacement and removal of Fischer-Tropsch catalyst from a gas-to-liquid (GTL) facility utilizing a Fischer-Trosch reaction. The invention further relates to a method for delivery, replacement and removal of Fischer-Tropsch catalyst from a transportable GTL facility.

### BACKGROUND OF THE INVENTION

Fischer-Tropsch processes for converting synthesis gas into higher carbon number hydrocarbons are well known. The hydrocarbon products of a Fischer-Tropsch synthesis generally include a wide range of carbon number, ranging from between about 1 and about 100. The end products which may be recovered from the Fischer-Tropsch synthesis product, following separation, hydroprocessing or other upgrading, include but are not limited to liquefied petroleum gas (“LPG”), naphtha, middle distillate fuels, e.g. jet and diesel fuels, and lubricant basestocks. Some of these end products, however, are more desirable than others for a variety of reasons, including for example, being marketable at a higher margin. Moreover, the desirability of an end product of a Fischer-Tropsch synthesis may be dependent upon geographic location. For example, the use of diesel fuel is significantly more widespread from a geographical perspective than the use of jet fuel.

While technological advances within the energy industry have made dramatic improvements in lowering the cost of finding, producing and refining oil, vast quantities of remote and stranded gas still wait to be developed. Gas to liquid (“GTL”) technologies may assist in developing and monetizing these resources. Such GTL technologies are especially critical to offshore applications given that about one-half of the world's stranded gas is located within submerged formations.

In conventional GTL processes, synthesis gas is generated from natural gas via partial oxidation with oxygen, requiring an air separation plant to provide the oxygen. In conventional approaches, nitrogen is eliminated from the synthesis gas stream as an unwanted inert. In an air-based system, however, synthesis gas is produced by oxidation of hydrocarbons using air or oxygen enriched air, rather than separated oxygen. This eliminates the expense, as well as the extra space requirement, of an air separation plant. It thus reduces capital costs, making possible plants with considerably smaller footprints, and also provides for a safer operating environment.

Stranded natural gas reserves also may produce condensates and liquefied petroleum gasses (LPGs), i.e. propanes and butanes, which may be recovered. Isolation of LPG components, with or without combination of Fischer-Tropsch produced LPGs, is not typically practiced in gas to liquid processes. However, failure to monetize LPG components further lowers the economic feasibility of accessing and producing stranded gas reserves.

As used herein, the term “stranded gas” means natural gas that cannot be economically delivered to market using current gas transportation methods or current commercial GTL processes. Stranded gas includes associated and flared/vented gas, and gas that is re-injected purely for regulatory compliance rather than for reservoir-pressure maintenance. Some of the factors that determine when a pipeline is profitable include resource volume, transport route, pipeline distance, regulatory environment, market size and demand growth. Excess reserves may be considered stranded where a paltry delivery rate is required to avoid oversupply of local markets. Negative economics may also arise from technical complexity or expense associated with recovering and/or gathering the gas.

Typically FT catalyst is delivered in oxide powder form or as active catalyst pastilles. In GTL facilities, and particularly in remote locations, the FT catalyst would typically be delivered in drums or supersacks. Although the amount of catalyst required is dependant upon the size of the FT reactor(s) and FT facility, quantities in the million pound area would not be unusual. Thus, a large number of drums or supersacks of solids would be required to deliver FT catalyst to an FT facility. Storage space would be required for the FT catalyst pre-loading as well as for spent or used FT catalyst. The oxide powder form of the FT catalyst must be activated prior to use, thereby requiring significant additional process equipment. Both the oxide powder and the pastillated catalyst would need to be manually loaded into a hopper to be fed into the process. Manually loading would expose personnel to catalyst and require additional personnel protective equipment.

### BRIEF DESCRIPTION OF THE DRAWINGS

FIG. 1 is a schematic diagram illustrating a first embodiment of the invention.

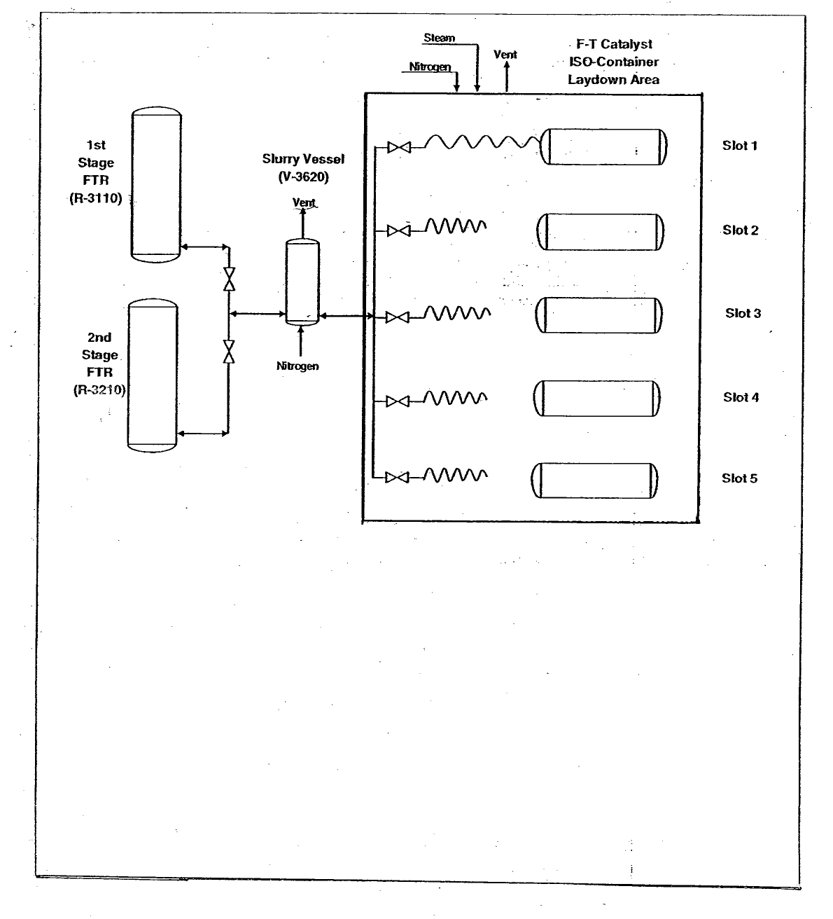

### DETAILED DESCRIPTION OF THE INVENTION

Embodiments of the invention simplify the method of delivering active Fischer-Tropsch (“FT”) catalyst to Fischer-Tropsch Reactors in either a marine-based or land-based FT facilities. Some embodiments of the invention provide a movable FT facility located in or near submerged formations, such as those found off-shore. Such a movable FT facility may be constructed upon, for example, ocean- or sea-going vessels, such as a floating production, storage and offloading (“FPSO”) platform, a floating storage and offloading (“FSO”) platform, gravity based structures, spar platforms, or tension leg platforms. In some embodiments of the invention, the movable platform is temporarily maintained in place by any of a number of methods, including without limitation, fixed turret, removable turret, conventional mooring systems, anchoring, and/or suction piles.

In other embodiments of the invention, a transportable inland barge may be used to access gas reserves in or near shallow water or onshore gas reserves that are near the coastline or other navigable waterway. In such instances, an FT facility may be constructed in whole or in part on such a transportable inland barge.

As used herein, the term “inland barge” means a barge which is transportable by lift ship or other form of barge dry haul and which is not suitable for towing or operation at sea or in any waters having wave action greater than that of Sea State 0 (as defined by Pierson-Moskowitz Sea Spectrum). It should be noted that the Sea State 0 is based upon wind speeds of around three (3) knots. However, as used herein, the term inland barge will include designs which may withstand wind loads of about 120 kilometers per hour or greater. The inland barge, however, may be towed within inland waters, such as rivers, lakes and intercoastal waterways. The inland barge is installed and then operated only in calm water. “Installed” is defined as either freely floating in confining moorings or fixed in a non-floating arrangement. Confining moorings will allow the barge to float on a water body allowing only uniform vertical motion with essentially no lateral or twisting motion. In some embodiments, a barge having jacking legs may be used and installation of the jacking legs installs the barge. As used herein, the term “calm water” means near shore, such as on pylons, beached, or in a natural or man-made inlet which may or may not be dammed and/or drained, or on a fixed platform if off-shore. The term “calm water” may also include inland waterways, such as rivers, lakes, ship channels, bayous, and intercoastal waterways which are protected from substantial natural wave action. Other methods of securing the barge in calm water include use of a flotation jacket surrounding the outer perimeter of the barge, anchoring, or installation of legs under the barge.

In other embodiments of the invention, the FT facility is constructed upon a land-based movable structure, such as a trailer, truckbed, rail car or platform, or other movable forms on which one or more section of an FT facility may be constructed and transported or moved from location to location.

Embodiments of the this invention also simplify the removal of spent FT catalyst from an FT reactor installed on a barge or FPSO. This invention eliminates the need for regeneration equipment to be installed on a barge or FPSO, eliminates the operators required to operate the catalyst regeneration equipment, and increases the barge or FPSO safety by minimizing the number of catalyst parcels handled on the FPSO.

The term “Cₓ”, where x is a number greater than zero, refers to a hydrocarbon compound having predominantly a carbon number of x. As used herein, the term Cₓ, may be modified by reference to a particular species of hydrocarbons, such as, for example, C₅ olefins. In such instance, the term means an olefin stream comprised predominantly of pentenes but which may have impurity amounts, i.e. less than about 10%, of olefins having other carbon numbers such as hexene, heptene, propene, or butene. Similarly, the term “Cₓ+” refers to a stream wherein the hydrocarbons are predominantly those having a hydrocarbon number of x or greater but which may also contain impurity levels of hydrocarbons having a carbon number of less than x. For example, the term C₁₅₊ means hydrocarbons having a carbon number of 15 or greater but which may contain impurity levels of hydrocarbons having carbon numbers of less than 15.

The term “Cₓ-C_{y}”, where x and y are numbers greater than zero, refers to a mixture of hydrocarbon compounds wherein the predominant component hydrocarbons, collectively about 90% or greater by weight, have carbon numbers between x and y. For example, the term C₅-C₉ hydrocarbons means a mixture of hydrocarbon compounds which is predominantly comprised of hydrocarbons having carbon numbers between 5 and 9 but may also include impurity level quantities of hydrocarbons having other carbon numbers.

Synthesis gas (or “syngas”) useful in producing a Fischer-Tropsch product useful in the invention may contain gaseous hydrocarbons, hydrogen, carbon monoxide and nitrogen with H₂:CO ratios from between about 0.8:1 to about 3.0:1. The hydrocarbon products derived from the Fischer-Tropsch reaction may range from methane to high molecular weight paraffinic waxes containing more than 100 carbon atoms. Operating conditions and parameters of an autothermal reactor for producing a syngas useful in the process of the invention are well known to those skilled in the art. Such operating conditions and parameters include but are not limited to those disclosed in U.S. Pat. Nos. 4,833,170; 4,973,453; 6,085,512; 6,155,039, the disclosures of which are incorporated herein by reference.

Fischer-Tropsch catalysts are also known in the art and include, those based upon for example, cobalt, iron, ruthenium as well as other Group VIIIB transition metals or combinations of such metals, to prepare both saturated and unsaturated hydrocarbons. The Fischer-Tropsch catalyst may also include a support, such as a metal-oxide support, including but not limited to silica, alumina, silica-alumina or titanium oxides. For example, a cobalt (Co) catalyst on transition alumina with a surface area of approximately 100-200 m²/g may be used in the form of spheres of 50-150 μm in diameter. The Co concentration on the support may be between about 5 wt % to about 30 wt %. Certain catalyst promoters and stabilizers may be used. The stabilizers include Group IIA or Group IIIB metals, while the promoters may include elements from Group VIII or Group VIIB. The Fischer-Tropsch catalyst and reaction conditions may be selected to be optimal for desired reaction products, such as for hydrocarbons of certain chain lengths or number of carbon atoms. Any of the following reactor configurations may be employed for Fischer-Tropsch synthesis: fixed bed, slurry bed reactor, ebullating bed, fluidizing bed, or continuously stirred tank reactor (“CSTR”). The FT reactor may be operated at a pressure from about 100 psia to about 800 psia and a temperature from about 300° F. to about 600° F. The reactor gas hourly space velocity (“GHSV”) may be from about 1000 hr⁻¹ to about 15000 hr⁻¹. Operating conditions and parameters of the FTR useful in the process of the invention are well known to those skilled in the art.

Embodiments of the invention simplify the method of delivering active FT catalyst to FT reactors. Embodiments of the invention further simplify the removal of spent, or used, FT catalyst from FT reactors. Moreover, embodiments of the method eliminate the requirement that an FT catalyst regeneration system be located in or proximate to an FT facility because the FT catalyst may be delivered in an active state.

Embodiments of the invention make use of bulk shipping containers, known as ISO containers to transport solid FT catalyst from a manufacturer to an FT facility. Typically, the catalyst would be in the reduced state and mixed with wax to prevent deactivation of the catalyst by exposure to air. The type of wax which may mixed with the active FT catalyst in embodiments of the invention is not limited to Fischer-Tropsch produced wax, but rather includes petroleum waxes, as well as vegetable and mineral waxes. ISO containers are available in various sizes. Most typical ISO containers have about a 6,300 gallon capacity and are typically designed for operation at about 87 psig and about 400° F. Any other type of shipping container which meets the pressure, temperature, and volume requirements of embodiments of the invention may also be utilized. Any such suitable container is referred to herein as an ISO container.

According to embodiments of the invention, the FT catalyst is reduced, and is therefore, active and ready for use in a FT Reactor. The FT catalyst may be reduced at a location near to or distant from the FT facility, including for example, at an FT catalyst manufacturer's location. The solid, active FT catalyst is then mixed with wax, creating a slurry. Typically the catalyst and wax are 50/50 wt % mixture, but other weight ratios are acceptable, ranging from about 20/80 wt % to about 80/20 wt %. Indeed, any weight ratio is acceptable so long as sufficient wax is used to prevent any significant deactivation of the FT catalyst. The FT catalyst/wax slurry is placed in ISO containers. The ISO containers can be conventionally shipped to the FT facility location, including the location of any non-transportable, transportable or movable FT facility such as marine based and land based FT facilities. In some embodiments of the invention, cranes are used to off-load the ISO containers from a transport vehicle or vessel onto a lay down area provided within or near the FT facility. In some embodiments of the invention, the ISO containers are heat traced and insulated.

Once the ISO containers are located in or near the FT facility, the FT catalyst/wax slurry may, but not in all circumstances, need to be heated and stirred and vented to flare or atmosphere. The FT catalyst/wax slurry may be stirred using either mechanical means or by passing inert gases through a distribution header. Once stirred and heated to sufficient temperature (typically about 250 to about 300° F.) the slurry may be transferred by mechanical pump or pressurized gas to a Slurry Vessel. The Slurry Vessel may be heat traced and may have an internal heating coil to further facilitate heating of the slurry to FT reactor temperatures (typically about 350 to about 450° F.). The Slurry Vessel is preferably designed for pressures in the range 450-550 psig and temperatures in the range about 450 to about 500° F., which would allow addition of heated pressurized FT catalyst/wax slurry directly to an operating FT reactor. In some embodiments of the invention, the FT catalyst/wax slurry is added to an FT Reactor that is shutdown or on standby. In some embodiments of the invention, the FT catalyst/wax slurry is pressurized with high pressure gas in the Slurry Vessel. The high pressure of the FT catalyst/wax slurry in the Slurry Vessel may be used to transfer the FT catalyst/wax slurry into an FT reactor at a lower pressure. Examples of gasses which may be used to pressurize the Slurry Vessel include inert gasses, such as noble gasses, as well as carbon dioxide, steam, and oxygen. Other gasses which may be used to pressurize the Slurry Vessel are known in the art. In other embodiments of the invention, a mechanical pump is used to transfer the FT catalyst/wax slurry from the Slurry Vessel to an FT reactor.

Some embodiments of the invention provide a method for removing spent or used FT catalyst from an FT Reactor. An FT catalyst/wax slurry in an FT Reactor may be pressurized as described above in connection with the Slurry Vessel by use of a high pressure gas and then pressure transferred to the Slurry Vessel. Alternatively, the FT catalyst/wax slurry in an FT reactor may be transferred into the Slurry Vessel by use of a mechanical pump. Following transfer of the FT catalyst/wax slurry into the Slurry Vessel, it may be stirred using either mechanical means or by passing inert gases through a distribution header to prevent settling of the catalyst. Depending upon the temperature of the FT catalyst/wax slurry transferred from an FT reactor into a Slurry Vessel, cooling maybe required prior to loading the FT catalyst/wax slurry into an ISO container. In some embodiments of the invention, the Slurry Vessel employs an internal cooling coil to cool the FT catalyst/wax slurry. In other embodiments, the FT catalyst/wax slurry is permitted to cool by convection with stirring. In some embodiments of the invention, the FT catalyst/wax slurry may be transferred by mechanical pump or pressurized gas to an ISO container. In preferred embodiments of the invention, the FT catalyst/wax slurry in the ISO container is then allowed to cool to a temperature sufficient for the FT catalyst/wax slurry to solidify. Embodiments of the invention further provide for the off-loading or removal of the ISO container(s) from the FT facility. The FT catalyst/wax slurry containing used or spent FT catalyst may then be subjected to metals reclamation procedures or may be disposed of by any acceptable disposal method.

One or more ISO container laydown areas and one or more Slurry Vessels may be used in embodiments of the invention.

The disclosures contained in the attached appendix are incorporated herein by reference. Embodiments of the invention may be further applied to or integrated with facilities and processes disclosed in the attached appendix.

We claim:

1. A method for handling catalyst, comprising the steps of:
providing a Fischer-Tropsch facility comprising a Fischer-Tropsch reactor;
providing a catalyst lay down area within or proximate to the Fischer-Tropsch facility, wherein the lay down area is provided with steam, nitrogen and venting;
delivering into the lay down area a transportation and storage container;
providing a slurry vessel; and
providing means for pumping a slurry between the container and the slurry vessel.

2. The method of claim 1, wherein the transportation and storage container contains a slurry of Fischer-Tropsch catalyst and wax.

3. The method of claim 1, further comprising the steps of:
providing means for pumping a slurry between the slurry vessel and the Fischer-Tropsch reactor.

4. The method of claim 1, wherein the slurry vessel includes means to heat the slurry.

5. The method of claim 4, wherein the means to heat a slurry is heat tracing.

6. The method of claim 4, wherein the means to heat a slurry is an internal heating coil.

7. The method of claim 1, wherein the transportation and storage container includes means to heat a slurry.

8. The method of claim 1, wherein the transportation and storage container includes means to stir a slurry.

9. The method of claim 1, where the slurry vessel includes means to cool the slurry.

10. The method of claim 9, wherein the means to cool the slurry is an internal cooling coil.
